(19) United States
(12) Patent Application Publication
(10) Pub. No.: US 2019/0150413 A1
(43) Pub. Date: May 23, 2019

(54) VE-PTP KNOCKOUT

(71) Applicant: MANNIN RESEARCH INC., Toronto (CA)

(72) Inventor: Susan QUAGGIN, Chicago, IL (US)

(21) Appl. No.: 16/098,843

(22) PCT Filed: May 4, 2017
(86) PCT No.: PCT/CA17/00120

§ 371 Date: Nov. 2, 2018

Related U.S. Application Data
(60) Provisional application No. 62/411,193, filed on Oct. 21, 2016.
(60) Provisional application No. 62/331,661, filed on May 4, 2016.

(51) Int. Cl. A01K 67/027; C12N 15/85; C12N 9/12

(52) U.S. Cl. CPC A01K 67/0276; C12N 15/8509; C12N 9/1205; C12N 2015/8536; C12Y 207/01112; A01K 2217/15; A01K 2227/105; A01K 2267/0306; A01K 2217/075

(57) ABSTRACT

This invention relates to glaucoma, and more particularly to use of VE-PTP-null allele to rescue from the glaucoma symptom of elevated intraocular pressure. This invention also relates to conditional knockout of VE-PTP to rescue from the glaucoma symptom of elevated intraocular pressure expressed in an Angiopoietin 1 and Angiopoietin 2 conditional knockout mouse. This invention also relates to the use of VE-PTP-null alleles.

### FIELD OF THE INVENTION

The invention relates to glaucoma, and more particularly to use of VE-PTP inhibition for rescue from glaucoma symptoms of elevated intraocular pressure.

### BACKGROUND OF THE INVENTION

About sixty million patients worldwide suffer from glaucoma, a devastating disease with no cure causing bilateral blindness in 8 million people worldwide. Current therapies only slow the progression of the disease. The most important risk factor leading to blindness is elevated intraocular pressure.

Schlemm's canal is a specialized vessel formed by a chain of cells around the eye. A mouse model is described in U.S. patent application Ser. No. 14/790,884 (publication No. US 2016/0000871 A1) in which double Angiopoietin 1/Angiopoietin 2 (“Angpt 1/Angpt 2”) knockout mice and Tie 2 knockout mice develop buphthalmos due to elevated intraocular pressure. Both Angpt 1/Angpt 2 double knockout mice and Tie2 knockout mice lack Schlemm's canal. Angiopoietin signaling has a dose-dependent effect on Schlemm's canal formation. Tie2 signaling (activation) has a dose-dependent effect on Schlemm's canal formation. Tie2 activation promotes canalogenesis in the Schlemm's canal, and factors which activate Tie2 include vascular endothelial-phosphotyrosine phosphatase (“VE-PTP”) inhibitors.

### SUMMARY OF THE INVENTION

In an embodiment of the present invention, there is a method of producing a mouse with reduced VE-PTP, comprising replacing at least one wild type VE-PTP allele with a VE-PTP-null allele.

In a further embodiment of the present invention, there is a method of producing a mouse with reduced VE-PTP, comprising replacing at least one wild type VE-PTP allele in a heterozygous Tie2 mouse with a VE-PTP-null allele.

The use of a VE-PTP-null allele introduced in a Tie2 heterozygous mouse decreases phenotypic expression of high intraocular pressure.

An embodiment of the present invention is a VE-PTP-null allele.

In an embodiment of the present invention, there is a mouse model comprising a mouse with a conditional triple knockout of Angiopoietin 1, Angiopoietin 2 and VE-PTP.

In a further embodiment of the present invention, there is a mouse model comprising a mouse with a conditional complete knockout of VE-PTP.

In an embodiment of the present invention, there is a method of producing a conditional triple knockout mouse, comprising replacing both wild type VE-PTP alleles with VE-PTP-null alleles in an Ang1/2 conditional knockout mouse.

In an embodiment of the present invention, there is a method of producing a VE-PTP conditional knockout mouse comprising replacing both wild type VE-PTP alleles with VE-PTP-null alleles.

In an embodiment of the present invention, the use of VE-PTP-null alleles to decrease high intraocular pressure in an Ang1/2 conditional knockout mouse.

In an embodiment of the present invention, the use of VE-PTP-null alleles to decrease high intraocular pressure in a mouse expressing a phenotype of high intraocular pressure.

In an embodiment of the present invention, the use of VE-PTP-null alleles in an Ang1/Ang2 conditional knockout mouse to eliminate phenotypic expression of high intraocular pressure.

### BRIEF DESCRIPTION OF THE FIGURES

FIG. 1 is a gel comparison of levels of Tie2 phosphorylation in control mice and VE-PTP heterozygous mice.

FIG. 2 is a chart comparison of Tie2 phosphorylation levels in control mice and VE-PTP heterozygous mice.

FIG. 3 is a comparison of intraocular pressure measurements in control mice, VE-PTP heterozygous mice, Tie2 heterozygous mice, and Tie2 heterozygous/VE-PTP heterozygous mice.

FIG. 4a is a comparison of phenotypic appearance of eyes and histological cross-section of Schlemm's canal in control mice, Ang1/2 conditional knockout mice and Ang1/2/VE-PTP conditional knockout (“3KO”) mice.

FIG. 4b is a comparison of intraocular pressure measurements in control mice, VE-PTP conditional knockout mice, Ang1/2 conditional knockout mice and Ang1/2/VE-PTP conditional knockout (“3KO”) mice.

### DETAILED DESCRIPTION

The construct, primers and components used are the same for these mice as the mice previously described in U.S. patent application Ser. No. 14/790,884 (publication No. US 2016/0000871 A1). From this reference it is known that A1A2Flox^{WBΔE16.5} (cK0 or conditional knockout) mice develop bilateral buphthalmos and that in Angiopoietin 1 and Angiopoietin 2 mouse conditional knockouts (“Ang1/2 conditional knockout mice”) Schlemm's canal is lacking and intraocular pressure (“IOP”) is increased.

There is a dose dependent role for Angpt/Tie2 signaling in canal formation. While Angpt1/2 double knockouts completely lack Schlemm's canal, Angpt1 knockout mice have only a hypomorphic phenotype with some canal tissue remaining. Angpt2 knockout alone has no effect, suggesting that Angpt1 is the primary ligand while Angpt2 can provide some compensation. intraocular pressure (“IOP”) measurements confirm these histological results, as Angpt1 knockout mice have elevated pressure (though not as elevated as double knockouts) while Angpt2 knockouts are normal. Tie2 activation (i.e. level of Angpt/Tie2 signaling) has a dose-dependent effect on canal formation.

An embodiment of the present invention is a VE-PTP null allele. An embodiment of the present in invention is a method of creating, and the mouse created, by introducing a VE-PTP-null allele into a control mouse or a Tie2 heterozygous mouse. An embodiment of the invention is a heterozygous VE-PTP mouse. In a further embodiment of the invention there is a heterozygous VE-PTP/heterozygous Tie2 mouse.

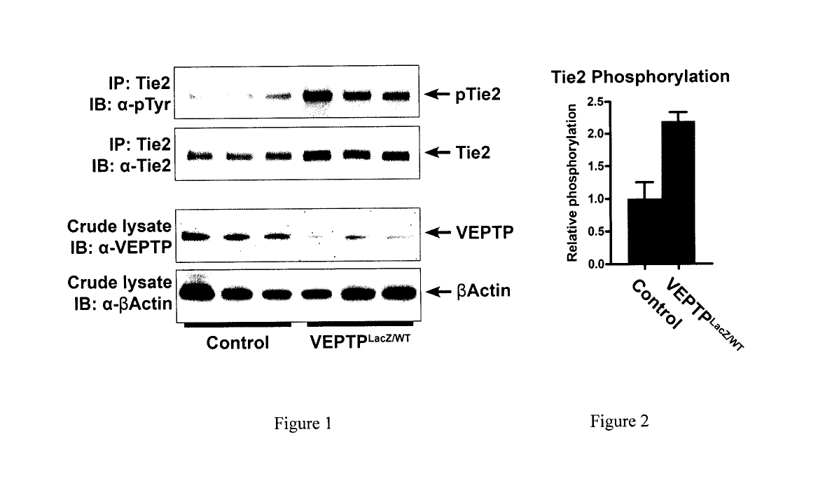

As shown in FIGS. 1 and 2 VE-PTP heterozygous mice have elevated Tie2 phosphorylation compared to control littermates.

FIG. 1 is a comparison between a control mouse and a heterozygous VE-PTP^{LacZ/WT} mouse showing that while the VE-PTP^{LacZ/WT} mouse has less VE-PTP than the control, the levels of pTie2 and Tie2 are higher in the VE-PTP^{LacZ/WT} mouse.

FIG. 2 shows that the phosphorylation of Tie2 in the control is less than half that of the Tie2 phosphorylation in the VE-PTP^{LacZ/WT} mouse.

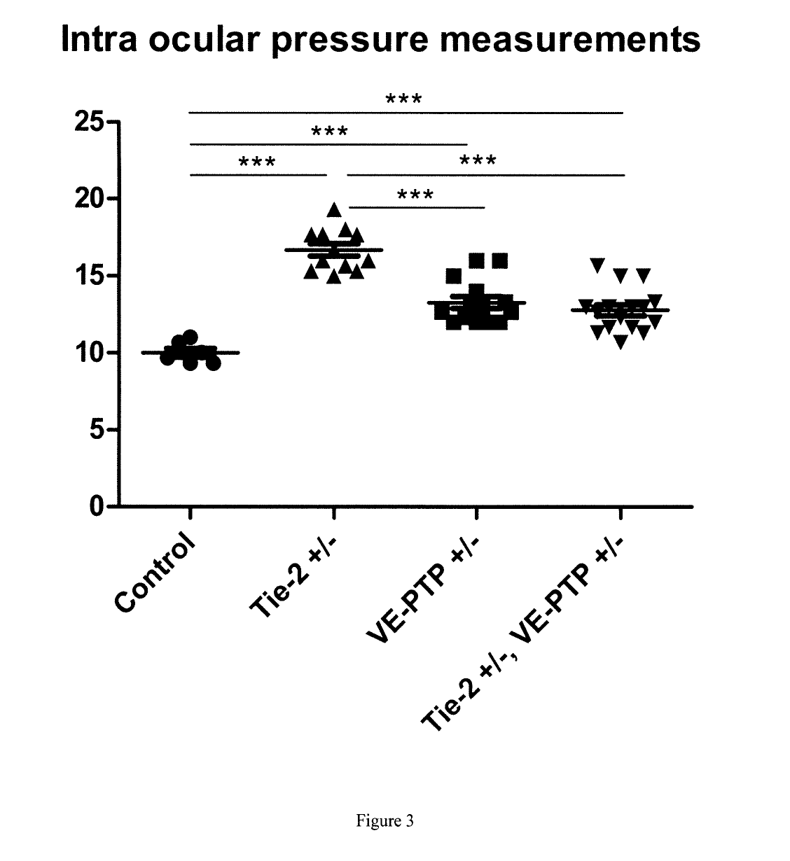

Introduction of a VE-PTP-null allele (VE-PTP heterozygosity) can rescue the developmental phenotype of the Tie2 heterozygous mice described above and prevent them from developing elevated IOP. FIG. 3 shows that the intraocular pressure (“IOP”) in a heterozygous VE-PTP mouse or combination Tie2/VE-PTP heterozygous mouse approaches the normal levels seen in the control, and is much less than the Tie2 knockout mouse.

The VE-PTP heterozygous mouse is derived in this embodiment from a WT-LacZ mouse from Charles River in which a VE-PTP-null allele was introduced to create a “VE-PTP^{LacZ/WT}” mouse.

This demonstrates that rescue from the glaucoma phenotype occurs with a VE-PTP-null allele introduction in a Tie2 heterozygous mouse.

The knockout of VE-PTP, in the context of mediated Tie2, or Tie2 heterozygous mice, rescues a mouse from the phenotype of increased LOP (i.e. with decreased VE-PTP, LOP is normal, and therefore mice don't have glaucoma symptoms of increased LOP).

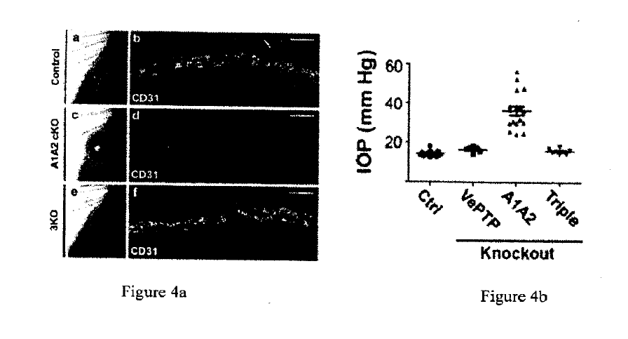

FIG. 4a is a comparison of phenotypic appearance of eyes and histological cross-section of Schlemm's canal in control mice, Ang1/2 conditional knockout mice and Ang1/2/VE-PTP conditional knockout (“3KO”) mice.

FIG. 4b is a comparison of intraocular pressure measurements in control mice, conditional VE-PTP knockout mice, Ang1/2 conditional knockout mice and Ang1/2/VE-PTP conditional knockout (“3KO”) mice.

Angpt1/2 conditional double knockout mice completely lack Schlemm's canal, and have protruding eyes compared to the control mice. Intraocular pressure (“IOP”) measurements confirm these phenotypic and histological results, since Angpt1/2 conditional knockout mice have elevated pressure while control and VE-PTP conditional knockout mice are normal.

As seen in FIGS. 4a and b, Ang1/2 conditional knockout mice which additionally are VE-PTP conditional knockouts approach the normal phenotype for eyes, histological appearance of Schlemm's canal and intraocular pressure measurements.

When Ang1/Ang2 and VEPTP are all conditionally knocked out in mice, there is a rescue of normal LOP, versus a “glaucomatous” mouse when only Ang1 and Ang2 are knocked out and VE-PTP is still present.

An embodiment of the present invention is a method of creating, and the mouse created, by introducing VE-PTP-null alleles into an Ang1/2 conditional knockout mouse. Another embodiment of the invention is a homozygous VE-PTP conditional knockout mouse.

As shown in FIG. 4b 3KO mice, VE-PTP conditional knockout mice and control mice have similar LOP compared to Ang1/2 conditional knockout mice which have elevated LOP.

Introduction of VE-PTP-null alleles (VE-PTP homozygosity) can rescue the developmental phenotype of the Ang1/2 conditional knockout mice described above and prevent them from developing elevated IOP.

This demonstrates that rescue from the glaucoma phenotype occurs with VE-PTP-null alleles introduced into an Ang1/2 conditional knockout mouse.

The knockout of VE-PTP, in the context of suppressed Tie2 or Ang1/2 conditional knockout mice, rescues a mouse from the phenotype of increased IOP (i.e. with elimination of VE-PTP, IOP is normal, and therefore mice don't have glaucoma symptoms of increased IOP).

What is claimed is:

1. A method of producing a mouse with reduced VE-PTP, comprising replacing at least one wild type VE-PTP allele with a VE-PTP-null allele.

2. The method in claim 1, in which the mouse is additionally a heterozygous Tie2 mouse.

3. The use of a VE-PTP-null allele introduced in a Tie2 heterozygous mouse to decrease phenotypic expression of high intraocular pressure.

4. A VE-PTP-null allele.

5. A mouse model comprising a mouse with a conditional triple knockout of Angiopoietin 1, Angiopoietin 2 and VE-PTP.

6. A mouse model comprising a mouse with a conditional complete knockout of VE-PTP.

7. A method of producing a conditional triple knockout mouse, comprising replacing both wild type VE-PTP alleles with VE-PTP-null alleles in an Ang1/2 conditional knockout mouse.

8. A method of producing a VE-PTP conditional knockout mouse comprising replacing both wild type VE-PTP alleles with VE-PTP-null alleles in a mouse.

9. The use of VE-PTP-null alleles to decrease high intraocular pressure in an Ang1/2 conditional knockout mouse.

10. The use of VE-PTP-null alleles to decrease high intraocular pressure in a mouse expressing a phenotype of high intraocular pressure.

11. The use of VE-PTP-null alleles in an Ang1/2 conditional knockout mouse to eliminate phenotypic expression of high intraocular pressure.
